(19) United States
(12) Patent Application Publication
(10) Pub. No.: US 2010/0092476 A1
(43) Pub. Date: Apr. 15, 2010

(54) PANCREATIC CANCER BIOMARKERS

(76) Inventors: Samir M. Hanash, Mercer Island, WA (US); Vitor M. Faca, Seattle, WA (US); Kenneth Song, Seattle, WA (US); Nabeel El-Bardeesy, Framingham, MA (US); Ronald A. DePinho, Brookline, MA (US)

Correspondence Address: MYERS BIGEL SIBLEY & SAJOVEC, PO BOX 37428, RALEIGH, NC, 27627, US

(21) Appl. No.: 12/514,860

(22) PCT Filed: Nov. 14, 2007
(86) PCT No.: PCT/US07/23788

§ 371 Date: Dec. 7, 2009

Related U.S. Application Data
(60) Provisional application No. 60/866,266, filed on Nov. 17, 2006.
(60) Provisional application No. 60/871,050, filed on Dec. 20, 2006.
(60) Provisional application No. 60/952,663, filed on Jul. 30, 2007.

(51) Int. Cl. A61K 39/395; C12Q 1/68

(52) U.S. Cl. 424/141.1; 435/6; 424/130.1

(57) ABSTRACT

The present invention provides a method of diagnosing, prognosing or screening for pancreatic cancer in a subject. The method may be carried out on a sample such as a blood or tissue sample collected from the subject. The is carried out by (a) detecting one or more markers in a biological sample of said subject, said markers selected from the markers set forth in Table 1 (e.g. one or more markers selected from the group set forth in Table 2, and/or the group consisting of ALCAM, TIMP-1, ICAM1, LCN2, REG1A, REG3, IGFBP4, TNFRSF1A and WFDC2); and (b) determining an altered level of said marker(s), said altered level indicating said subject may be afflicted with or at risk of developing pancreatic cancer. Kits useful for carrying out the methods are also described.

### RELATED APPLICATION INFORMATION

This application claims priority under 35 U.S.C. 119(e) from U.S. Provisional Patent Application Ser. Nos. 60/866,266, filed Nov. 17, 2006; 60/871,050, filed Dec. 20, 2006; and 60/952,663, filed Jul. 30, 2007, the disclosures of which are incorporated by reference herein in their entireties.

### FIELD OF THE INVENTION

The present invention concerns methods of screening for or detecting pancreatic cancer in subjects, including screening for or detecting blood-based biomarkers.

### BACKGROUND OF THE INVENTION

Cancer of the pancreas, the fourth leading cause of cancer death in the United States, often eludes early detection due to a lack of specific symptoms and limitations in current diagnostic methods. The most common type of pancreatic cancer is pancreatic ductal adenocarcinoma, accounting for more than 85% of pancreatic tumors. Pancreatic ductal adenocarcinoma is also referred to as pancreatic adenocarcinoma, or simply pancreatic cancer. The 5-year survival rate of patients diagnosed with this disease is a mere 3%, and the median survival is less than 6 months. See Bardeesy and DePinho (2002) Nature Rev. 2:897-909.

While our understanding of the molecular pathogenesis of common types of human cancer has advanced considerably, the development of effective strategies for cancer diagnosis and treatment have lagged across a number of tumor types. Early stage cancers are generally more easily cured, often through resection, and early diagnosis through screening has led to improved survival in patients (Etzioni, et al. (2003) Nat. Rev. Cancer 3:243). The vast dynamic range of protein abundance in plasma and the likely occurrence of tumor-derived proteins in the lower range of protein abundance represent major challenges in the application of proteomic-based strategies for biomarker identification. Recent experience in comprehensive profiling of plasma proteins indicates that low abundance proteins may be identified with high confidence following extensive plasma fractionation and with the use of high-resolution mass spectrometry (States, et al. (2006) Nat. Biotechnol. 24:333). At the same time, the vastly complex datasets generated by these extensive plasma proteome analyses have presented challenges in the prioritization of candidates for subsequent in-depth validation.

Blood-based biomarkers hold significant potential to transform the practice of early cancer detection and patient management (Etzioni, et al. (2003) Nat. Rev. Cancer 3:243). Despite significant technological advances in proteomics and computational science, the complexity and heterogeneity of the human serum proteome have presented significant challenges in the identification of protein changes associated with tumor development (Hanash (2003) Nature 422:226). Refined genetically engineered mouse (GEM) models of human cancer have been shown to faithfully recapitulate the molecular, biological and clinical features of human disease (Sweet-Cordero, et al. (2005) Nat. Genet. 37:48; Aguirre, et al. (2003) Genes Dev. 17:3112; Bardeesy, et al. (2006) Proc. Natl. Acad. Sci. USA 103:5947; Hingorani, et al. (2005) Cancer Cell 7:469).

Recent genomic analysis of human and mouse cancers has revealed significant concordance in chromosomal aberrations and molecular profiles, establishing cross-species analyses as a highly effective filter in the identification of genes and loci embedded within extremely complex cancer genomes (Sweet-Cordero, et al. (2005) Nat. Genet. 37:48; Zender, et al. (2006) Cell 125:1253). Such observations point to the potential utility of these GEM models in the identification and prioritization of candidate diagnostic markers among the highly complex serum proteome of human cancers. In this regard, it is notable that GEM models afford defined stages of tumor development, homogenized breeding and environmental conditions, and standardized blood sampling thereby limiting biological heterogeneity. The concept that plasma from GEM models of cancer contains tumor-derived proteins that may be relevant as candidate markers for human cancer is attractive as suggested by SELDI scanning technology, but remains untested as no novel markers have been identified using such models and methods (Hingorani, et al. (2003) Cancer Cell 4:437).

### SUMMARY OF THE INVENTION

The present invention provides a method of diagnosing, prognosing or screening for pancreatic cancer in a subject. The method may be carried out on a sample such as a blood or tissue sample collected from the subject.

The method of diagnosing, prognosing or screening for pancreatic cancer comprises (a) detecting one or more markers in a biological sample of said subject, said markers selected from the markers set forth in Table 1 (e.g. one or more markers selected from the group set forth in Table 2, and/or the group consisting of ALCAM, TIMP-1, ICAM1, LCN2, REG1A, REG3, IGFBP4, TNFRSF1A and WFDC2); and (b) determining altered level of said marker(s), said altered level indicating said subject may be afflicted with or at risk of developing pancreatic cancer.

Another aspect of the invention is the use of, or a kit comprising, a means of diagnosing, prognosing or screening for pancreatic cancer markers as described herein for carrying out a method of detecting a possible affliction with, or risk of developing, pancreatic cancer as described herein.

In some embodiments, the markers described herein can be detected in combination with, or concurrently with the detection of, the known pancreatic marker CA19.9, to facilitate the earlier diagnosis or detection of pancreatic cancer.

A further aspect of the present invention is a method of treating pancreatic cancer in a subject in need thereof, comprising: administering said subject a therapeutic antibody in an amount effective to treat said cancer, wherein said therapeutic antibody specifically binds to a marker set forth in Table 1 or Table 2 herein (e.g., a marker selected from the group consisting of ALCAM, TIMP-1, ICAM1, LCN2, REG1A, REG3, IGFBP4, TNFRSF1A and WFDC2).

A still further aspect of the present invention is the use of a therapeutic antibody as described herein for the preparation of a medicament for treating pancreatic cancer.

The foregoing and other objects and aspects of the present invention are explained in greater detail in the drawings herein and the specification set forth below.

### BRIEF DESCRIPTION OF THE DRAWINGS

FIG. 1. PanIN and PDAC mice for plasma proteomic analyses. Pools of Pdx1-Cre Ink4a/Arf^{lox/lox} and Kras^{G12D} Ink4a/Arf^{lox/lox} mice at 5.5 weeks and 7 weeks of representing PanIN and PDAC lesions, respectively, were sacrificed along with corresponding age- and sex-matched controls to yield 1 ml of plasma per disease phenotype for further analysis.

FIG. 2. Identification of low abundant proteins in mouse plasma. FIG. 2A, The number of MS2-events acquired in the experiment performed for early stage pancreatic cancer mouse plasma protein (PanIN) was correlated with protein plasma concentration reported by Rules-Based Medicine. As an approximation, protein concentration was estimated with the correlation (n. MS2 events=(0.623*Protein Concentration)+0.0625). The 23 proteins used for this estimation were: SERPINA1B, ADIPOQ, A2M, APOA1, APOC3, APOH, B2M, C3, CEACAM1, CRP, FABP1, F7, FTL1, FGB, HP, ICAM1, IGF1, MB, SERBP1, TIMP1, VCAM1, VWF. FIG. 2B, An inverse relationship occurs between the total number of proteins identified and number of MS2 events observed.

FIG. 3. Protein Identification from PanIN and PDAC analyses. Proteins identified in PanIN and PDAC analyses were further prioritized for biomarker candidacy. Up-regulated proteins were those with neoplasm/normal ratio ≧2 with associated p-value <0.05. Exclusion of liver proteins was based on prior mouse liver proteomic studies. Concordant gene expression refers to mouse PDAC/normal mRNA expression ≧2. *FDR refers to false discovery rate.

FIG. 4. Immunohistochemical analysis (IHC) of candidate PDAC biomarkers showing concordance between mouse and human tissue. FIGS. 4A, IHC of mouse pancreatic tissue. Panels A-C show PTPRG expression. Note islet staining in normal pancreas

(Panel A) and membranous staining in PanIN and PDAC epithelium (Panels B-C). Panels D-F show TNC expression. Note lack of staining in normal pancreatic tissue (Panel D) and strong expression present in stroma of PanIN (Panel E) and PDAC (Panel F). Panels G-I show ALCAM expression. Note membranous staining of the normal pancreatic acinar and ductal cells (Panel G) with increased staining present in the PanIN epithelium (Panel H) and

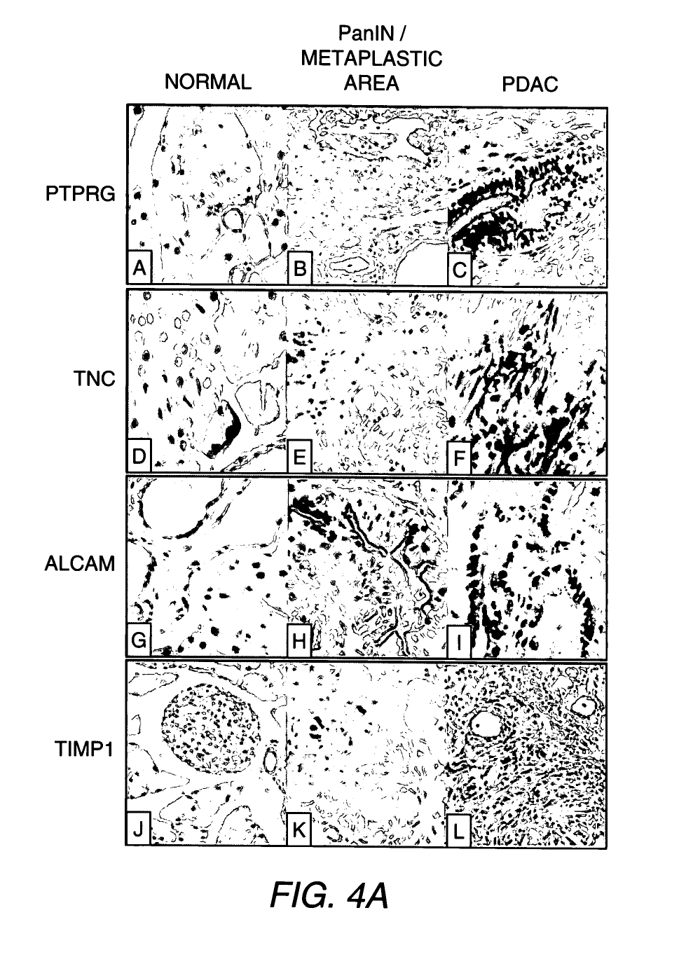

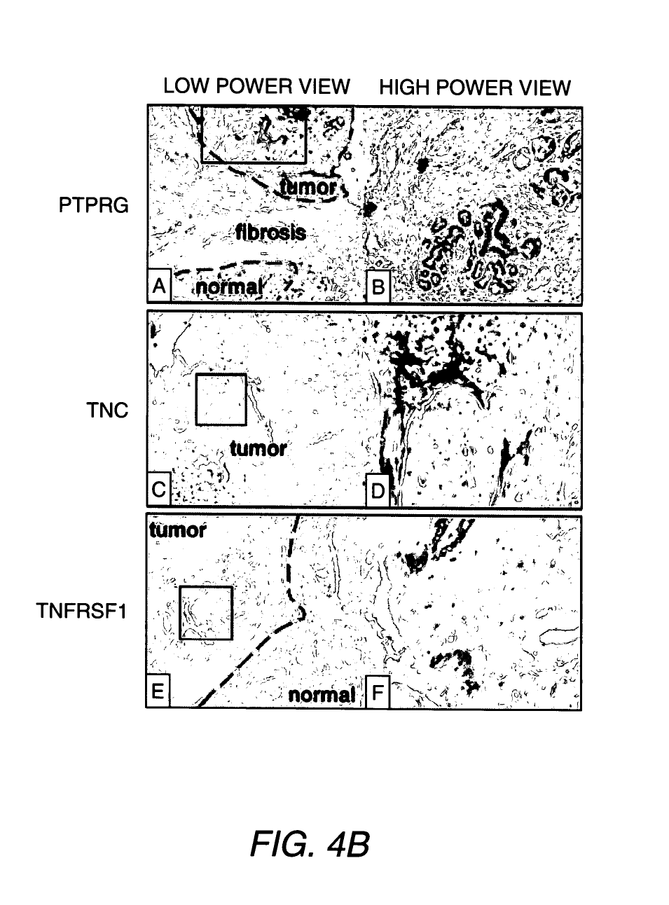

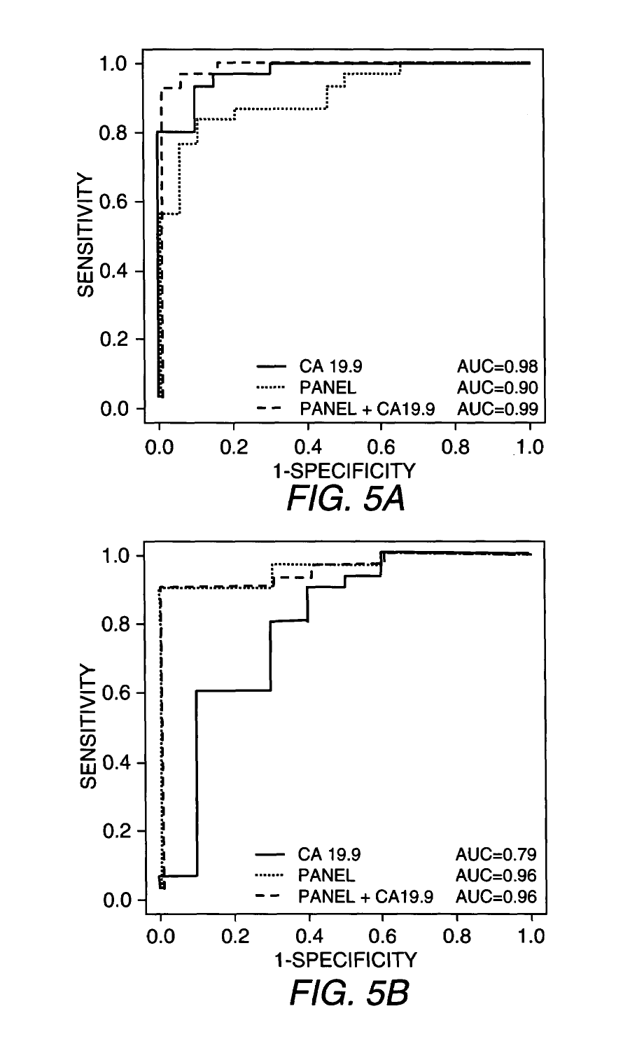

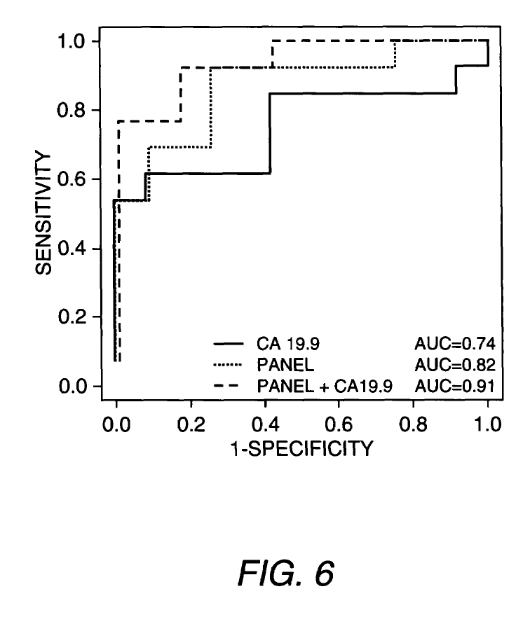

PDAC cells (Panel I). Panels J-L show TIMP1 expression. Note lack of staining in normal pancreatic tissue (Panel J and staining observed in association with acinar-ductal metaplasia (Panel K) and both PDAC stromal and tumor cells (Panel L). Panels D, E, F, G, H, and I: Magnification 400×; and Panels A, B, C, J, K, and L: Magnification 200×. FIG. 4B, IHC of human pancreatic tissue. Panels A-B show PTPRG expression. Note membranous staining in PDAC epithelium and absence of staining in normal pancreas. Panels C-D show TNC expression. Note expression in PDAC stroma. Panels E-F show TNFRSF 1 expression. Note membranous staining in PDAC epithelium; normal pancreatic tissue is negative. Dashed lines subdivide different histology of the tissue analyzed and boxes indicate the adjacent magnified region. Panels A, C, and E: Magnification 100×; Panel B: Magnification 200×; and Panels D and F: Magnification 400×.

FIG. 5. ROC performance of CA19.9 and candidate marker panel for newly diagnosed patients. FIG. 5A depicts subjects with cancer versus healthy subjects and

FIG. 5B depicts subjects with cancer versus subjects with pancreatitis. For this composite analysis, measurements of ALCAM, ICAM1, LCN2, TIMP1, REG1A, REG3 and IGFBP4 were employed. Standardization procedure and composite marker receiving operator curves (ROC) were generated without fitting by inclusion of all tested candidate markers. Specimens from healthy and pancreatitis subjects were obtained from the same institution and with the same protocol for blood collection.

FIG. 6 ROC performance of CA19.9 and candidate marker panel at pre-diagnosis. The composite analysis used data obtained for LCN2, TIMP1, REG1A, REG3 and IGFBP4. Standardization procedure and ROC for the composite marker panel were done without fitting by inclusion of all tested candidate markers.

### DETAILED DESCRIPTION OF THE PREFERRED EMBODIMENTS

1. Definitions.

“Altered level” or “altered levels” as used with respect to marker proteins herein refers to an increased level (e.g., a one or two fold increase, or more) or a decreased level (e.g., a one or two-fold decrease, or more) in the quantity of one or more marker proteins detectable in or via a biological sample from a subject, as compared to a level or levels of one ore more marker proteins in a corresponding subject not afflicted with a pancreatic disease such as pancreatic cancer.

“Biological sample” as used herein refers to any material taken from the body of a subject that may carry the target compound or compounds of the tests described herein, including both tissue samples and biological fluids such as blood samples, saliva samples, urine samples, etc.

“Blood sample” as used herein refers to whole blood or any fraction thereof that may contain detectable levels of marker proteins therein (if marker proteins are present in the whole blood sample from which said fraction is obtained), and in particular embodiments refers to a blood sera or blood plasma sample.

“Diagnosing”, “prognosing” or “screening” as used herein means providing an indication that a subject may be afflicted with or at risk of developing a disease, particularly a pancreatic disease such as pancreatic cancer, and includes other terms such as screening for a disease, providing a risk assessment for disease, etc. It will be appreciated that no such technique is perfect and that such diagnosis, prognosis or the like may be confirmed by other procedures such as physical examination, imaging, histological examination of tissue samples, etc. The term “prognosing” as used herein includes providing an assessment or indication of disease in response to treatment (such as surgical, radiation therapy, chemotherapy, and combinations thereof) after initial diagnosis, as an indication of the efficacy of the treatment, risk of the disease returning, severity of disease following treatment, or the like.

“Marker protein”, “marker” or “biomarker” as used herein refers to any protein that can be detected, directly or indirectly (e.g., via an analog, metabolite, fragment or breakdown product) in a biological sample from a subject, an increase or decrease of the amount of which, compared to amounts found in similar subjects without disease, is indicative of the presence or risk of pancreatic cancer in a subject. Marker proteins described herein include any protein listed in Table 1 (and in some embodiments particularly set forth in Table 2) herein. The analog, metabolite, fragment or breakdown product of the marker protein may or may not possess the functional activity of the marker protein listed.

“CA19.9” is a known marker protein for pancreatic cancer, and can be detected in accordance with known techniques, including but not limited to those described in U.S. Pat. Nos. 6,716,595; 6,248,063; and 5,126,243.

“Pancreatic cancer” as described herein refers to any type of cancerous or pre-cancerous tissues arising from normal tissues of the pancreas, including, but not limited to, pancreatic ductal adenocarcinoma or pancreatic adenocarcinoma. Other types of pancreatic tumors include acinar-cell carcinoma, pancreatic endocrine tumours, and serous cystadenoma. See Bardeesy and DePinho (2002) Nature Rev. 2:897-909.

“Panel test” as described herein refers to a group of individual laboratory tests that are related in some way, including, but not limited to, the medical condition they are designed to detect (e.g., pancreatic cancer), the specimen type (e.g., blood), and the methodology employed by the test (e.g., detection of altered level of a target protein or proteins).

“Subjects” as described herein are generally human subjects and includes “patients”. The subjects may be male or female and may be of any race or ethnicity, including but not limited to Caucasian, African-American, African, Asian, Hispanic, Indian, etc. The subjects may be of any age, including newborn, neonate, infant, child, adolescent, adult, and geriatric. Subjects may also include animal subjects, particularly mammalian subjects such as dog, cat, horse, mouse, rat, etc., screened for veterinary medicine or pharmaceutical drug development purposes. Subjects include but are not limited to those who may have, possess, have been exposed to, or have been previously diagnosed as afflicted with one or more risk factors for pancreatic cancer. Risk factors include age, gender, race, smoking, diet, obesity, diabetes, chronic pancreatitis, work exposure, family history, and stomach problems. These risk factors may be considered in combination with the disclosed methods of detecting pancreatic cancer for a diagnosis, prognosis or screening. The disclosed methods of detecting pancreatic cancer for a diagnosis, prognosis or screening may also be used in combination with other diagnostic methods, including, but not limited to, scanning of the pancreas by an ultrasound or CT scan of the abdomen, detection of bilirubin and other substances, physical signs of jaundice, performing a biopsy, and screening for other markers or other indicators of the possibility of pancreatic cancer, e.g., mutations in KRAS, CDKN2A, TP53 and SMAD4/DPC4. See Bardeesy and DePinho (2002) Nature Rev. 2:897-909. Those skilled in the art will appreciate that this listing of other methods of detecting pancreatic cancer for a diagnosis, prognosis or screening is by no means exhaustive, and is but a small sampling of the other possible diagnostic methods that can easily be combined with the disclosed methods for purposes of diagnosis, prognosis or screening for pancreatic cancer.

While the following description focuses primarily on pancreatic cancer, it will be appreciated that the present invention may also be utilized in connection with other pancreatic diseases as noted above.

The disclosures of all United States patent references cited herein are to be incorporated herein by reference in their entirety.

2. Assay Procedures.

The step of collecting a sample can be carried out either directly or indirectly by any suitable technique. For example, a blood sample from a subject can be carried out by phlebotomy or any other suitable technique, with the blood sample processed further to provide a serum sample or other suitable blood fraction.

The step of determining the presence of an altered level of a marker protein in the sample, and/or depressed level of a marker protein in the sample, can also be carried out either directly or indirectly in accordance with known techniques, including, but not limited to, mass spectrometry, chromatography, electrophoresis, sedimentation, isoelectric focusing, and antibody assay. See, e.g., U.S. Pat. No. 6,589,748; U.S. Pat. No. 6,027,896. Marker proteins may also be identified by two-dimensional electrophoresis (2-D electrophoresis). 2D-electrophoresis is a technique comprising denaturing electrophoresis, followed by isoelectric focusing; this generates a two-dimensional gel (2D gel) containing a plurality of separated proteins. For an example of a preferred means of carrying out 2D-electrophoresis to identify marker proteins, see, e.g. WO 98/23950; U.S. Pat. No. 6,064,654 and U.S. Pat. No. 6,278,794. Briefly, spots identified in a 2D gel are characterized by their isoelectric point (pI) and apparent molecular weight (MW) as determined by 2D gel electrophoresis. Altered levels of marker proteins in a first sample or sample set with respect to a second sample or sample set can be determined when 2D gel electrophoresis gives a different signal when applied to the first and second samples or sample sets. Altered levels of marker proteins may be present in first sample or sample sets at increased, elevated, depressed or reduced levels as compared to the second sample or sample sets. By “increased level” it is meant (a) any level of a marker protein when that marker protein is not present in a normal subject without pancreatic cancer, as well as (b) an elevated level (e.g., a two- or three-fold increase in detected quantity) of marker protein or a particular isoform of a marker protein when that protein or a particular isoform is present in a normal subject without pancreatic cancer. By “depressed level” it is meant (a) an absence of a particular marker protein or isoform of a particular marker protein when that marker protein is present in a normal subject without pancreatic cancer, as well as (b) a reduced level (e.g., a two- or three-fold reduction in detected quantity) of a marker protein or isoform of a marker protein when that protein or isoform is present in a normal subject without pancreatic cancer. In general, the steps of (a) assaying a sample for an elevated level of a marker protein and/or depressed level of a marker protein, and (b) correlating an elevated level of a marker protein and/or a depressed level of a marker protein in said sample with pancreatic cancer, can be carried out in accordance with known techniques or variations thereof that will be apparent to persons skilled in the art. See, e.g., U.S. Pat. No. 4,940,658 to Allen et al.

Signals obtained upon analyzing a biological sample or sample set from subjects having pancreatic cancer relative to signals obtained upon analyzing a biological sample or sample set from normal subjects without pancreatic cancer will depend upon the particular analytical protocol and detection technique that is used. Accordingly, the invention contemplates that each laboratory will establish a reference range for each marker protein identifier (e.g., pI and/or MW) in normal subjects without pancreatic cancer according to the analytical protocol and detection technique in use, as is conventional in the diagnostic art.

Antibody assays (immunoassays) may, in general, be homogeneous assays or heterogeneous assays. In a homogeneous assay the immunological reaction usually involves the specific antibody, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, the antibody to the marker protein and a system or means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody that binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Those skilled in the art will be familiar with numerous specific immunoassay formats and variations thereof, which may be useful for carrying out the methods disclosed herein. See generally Maggio (1980) Enzyme-Immunoassay, CRC Press, Inc., Boca Raton, Fla.; see also U.S. Pat. No. 4,727,022 to Skold et al. entitled “Methods for Modulating Ligand-Receptor Interactions and their Application,” U.S. Pat. No. 4,659,678 to Forrest et al., U.S. Pat. No. 4,376,110 to David et al., U.S. Pat. No. 4,275,149 to Litman et al., U.S. Pat. No. 4,233,402 to Maggio et al., and U.S. Pat. No. 4,230,767 to Boguslaski et al.

Antibodies for immunoassays can be polyclonal or monoclonal antibodies, Fab fragments, humanized antibodies and chimeric antibodies (including fragments thereof) and can be produced in accordance with known techniques, based on one or more marker protein. For example, monoclonal antibodies may be produced in a hybridoma cell line according to the techniques of Kohler and Milstein (1975) Nature 265:495-97. Monoclonal Fab fragments may be produced in Escherichia coli from the known sequences by recombinant techniques known to those skilled in the art. See, e.g., Huse (1989) Science 246:1275-81 (recombinant

Fab techniques). Polyclonal antibodies can be produced in animals such as goats, rabbits and horses by administration of one or more marker protein, optionally in combination with an adjuvant, as an immunogen, optionally administering booster doses thereof, and collecting the polyclonal antibodies from the animal.

Kits for diagnosis, prognosis or screening for pancreatic cancer are also provided, and in some embodiments include at least one biochemical material and/or reagent, such as buffers and/or binding partners that are capable of specifically binding with one or more marker proteins from Table 1 (and in some embodiments particularly set forth in Table 2). These can provide a means for determining binding between the biochemical material and one or more marker proteins, whereby at least one analysis to determine a presence of one or more marker proteins, analyte thereof, or a biochemical material specific thereto, is carried out on a biological sample. Optionally such analysis or analyses may be carried out with the additional use of detection devices for immunoassay, chromatography, spectrometry, electrophoresis, sedimentation, isoelectric focusing, or any combination thereof. Analysis may be carried out on a single sample or multiple samples. In addition, the kit may optionally include instructions for performing the method or assay. Additionally the kit may optionally include depictions or photographs that represent the appearance of positive and negative results. In some embodiments, the components of the kit may be packaged together in a common container.

3. Panel Tests.

The marker proteins described herein can be detected individually or in panels with one another or other additional markers for pancreatic cancer such as described above. Where used in a panel test, the levels of the various markers are optionally but preferably tested from the same biological sample obtained from the subject (e.g., by detecting the quantities or amounts of various proteins in the same blood sample obtained from a patient). When combined in a panel test, the panel test may include determining an altered level for each of 2, 3, 4, 5, or 6 different marker proteins, up to 38 or more different proteins (e.g., a panel of some or all proteins set forth in Table 1 below (and in some embodiments particularly set forth in Table 2)). The combination of multiple marker proteins in a panel test serves to reduce the number of false positives and false negatives should an aberrant value for one particular member of the panel be found.

Kits for diagnosis, prognosis or screening for pancreatic cancer are also provided, and in some embodiments include at least one biochemical material and/or reagent, such as buffers and/or binding partners, that is capable of specifically binding with one or more marker proteins from Table 1 (and in some embodiments particularly set forth in Table 2) included in a panel. These can provide a means for determining binding between the biochemical material and one or more marker proteins of the panel, whereby at least one analysis to determine a presence of one or more marker proteins, analyte thereof, or a biochemical material specific thereto, is carried out on a biological sample. Optionally such analysis or analyses may be carried out with the additional use of detection devices for immunoassay, chromatography, spectrometry, electrophoresis, sedimentation, isoelectric focusing, or any combination thereof. Analysis may be carried out on a single sample or multiple samples. In addition, the kit may optionally comprise instructions for performing the method or assay. Additionally the kit may optionally comprise depictions or photographs that represent the appearance of positive and negative results. In some embodiments, the components of the kit may be packaged together in a common container.

4. Therapeutic Antibodies.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁷Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, 89Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, and ²¹²Pb.

“Chemotherapeutic agent” as used herein includes but is not limited to methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine

“Cytotoxic agent” as used herein includes but is not limited to ricin (or more particularly the ricin A chain), aclacinomycin, diphtheria toxin, Monensin, Verrucarin A, Abrin, Vinca alkaloids, Tricothecenes, and Pseudomonas exotoxin A.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms, rather “treatment” and “prevention” refer to any type of treatment that imparts a benefit to a patient afflicted with a disease, including improvement in the condition of the patient (e.g., in one or more symptoms), delay in the progression of the disease, etc.

“Treatment effective amount” as used herein means an amount of the antibody sufficient to produce a desirable effect upon a patient inflicted with lymphoma, including improvement in the condition of the patient (e.g., in one or more symptoms), delay in the progression of the disease, etc.

The present invention is primarily concerned with the treatment of human subjects, including male and female subjects and neonatal, infant, juvenile, adolescent, adult, and geriatric subjects, but the invention may also be carried out on animal subjects, particularly mammalian subjects such as mice, rats, dogs, cats, livestock and horses for veterinary purposes, and for drug screening and drug development purposes.

The disclosures of all United States patent references cited herein are to be incorporated herein by reference in their entirety.

Antibodies used for therapy (i.e., in a method of combating cancer) may be polyclonal or monoclonal antibodies per se or monoclonal antibodies coupled to a therapeutic agent. Such antibodies are sometimes referred to herein as therapeutic antibodies.

Any therapeutic agent conventionally coupled to a monoclonal antibody may be employed, including (but not limited to) radionuclides, cytotoxic agents, and chemotherapeutic agents. See generally Monoclonal Antibodies and Cancer Therapy (R.

Reisfeld and S. Sell Eds. 1985)(Alan R. Liss Inc. N.Y.). Therapeutic agents such as radionuclides, cytotoxic agents and chemotherapeutic agents are described above, and also described in U.S. Pat. Nos. 6,787,153; 6,783,760; 6,676,924; 6,455,026; and 6,274,118.

Therapeutic agents may be coupled to the antibody by direct means or indirect means (e.g., via a chelator) by any suitable technique, including but not limited to those described in U.S. Pat. Nos. 6,787,153; 6,783,760; 6,676,924; 6,455,026; and 6,274,118. Therapeutic agents may be coupled or conjugated to the antibody by the Iodogen method or with N-succinimidyl-3-(tri-n-butylstanyl)benzoate (the “ATE method”), as will be apparent to those skilled in the art. See, e.g., Zalutsky and Narula (1987) Appl. Radiat. Isot. 38:1051.

Blocking antibodies can also be administered in conjunction with antibody therapy, as described in Abrams et al., U.S. Pat. No. RE38,008.

Formulations. The therapeutic antibodies and (if desired) blocking antibodies will each generally be mixed, prior to administration, with a non-toxic, pharmaceutically acceptable carrier substance (e.g., normal saline or phosphate-buffered saline), and will be administered using any medically appropriate procedure, e.g., parenteral administration (e.g., injection) such as by intravenous or intra-arterial injection.

The antibody compounds described above may be formulated for administration in a pharmaceutical carrier in accordance with known techniques. See, e.g., Remington, The Science and Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a liquid and is preferably formulated with the compound as a unit-dose formulation which may contain from 0.01 or 0.5% to 95% or 99% by weight of the active compound.

As discussed further below, the therapeutic antibodies may optionally be administered in conjunction with other, different, active compounds useful in the treatment of the disorders or conditions described herein (e.g., chemotherapeutics). The other compounds may be administered concurrently. As used herein, the word “concurrently” means sufficiently close in time to produce a combined effect (that is, concurrently may be simultaneously, or it may be two or more administrations occurring before or after each other).

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient.

Blocking and therapeutic antibodies may be provided in lyophilyzed form in a sterile aseptic container or may be provided in a pharmaceutical formulation in combination with a pharmaceutically acceptable carrier, such as sterile pyrogen-free water or sterile pyrogen-free physiological saline solution.

Administration. The blocking antibodies and therapeutic antibodies may be administered by any medically appropriate procedure, e.g., normal intravenous or intra-arterial administration, injection into the cerebrospinal fluid). In certain cases, intradermal, intracavity, intrathecal or direct administration to the tumor or to an artery supplying the tumor is advantageous.

Dosage of the blocking antibody will depend, among other things, the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

Dosage of the therapeutic antibody will likewise depend, among other things, the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

The present invention is explained in greater detail in the following non-limiting examples.

### Example 1

### Materials and Methods

Mice and Plasma Pooling. Pdx1-Cre Ink4a/Arf lox/lox and KrasG12D Ink4a/Arf lox/lox mice (Aguirre, et al. (2003) Genes Dev. 17:3112) were bred and euthanized at 5.5 and 7 weeks, respectively. Mice exhibited a range of PanIN (5.5 weeks), encompassing mainly PanIN-1, and PanIN-2 stages. Only Kras Ink4a/Arf mice presenting granular histopatology, the most common pathology observed in human cases, were used to represent the PDAC (7 weeks) group. Plasma from PanIN and PDAC mice and corresponding controls based on sex and age were pooled for further analysis.

Sample Immunodepletion and Isotopic Labeling. Plasma pools were immunodepleted of the top three most abundant proteins (Albumin, IgG, and Transferrin). Following immunodepletion, samples were labeled with acrylamide isotopes. PanIN and PDAC plasma pools were combined with their corresponding control plasma pool for further fractionation.

Protein Fractionation. Immunodepleted and isotope labeled plasma pools underwent conventional two-dimensional sequential protein fractionation using anion exchange chromatography followed by reverse phase chromatography.

Mass Spectrometry Analysis and Protein Identification and Quantitation. For protein identification, fractionated samples were submitted to in-solution digestion with trypsin followed by LC-MS/MS analysis in a LTQ-FTT™ mass spectrometer (Thermo-Finnigan, San Jose, Calif.) coupled to a nanoAcquity™ nanoflow chromatography system (Waters, Milford, Mass.). Data was further processed and analyzed using CPAS (Rauch, et al. (2006) J. Proteome Res. 5:112-21). The database used for protein identification was mouse IPI version.3.12. Relative quantitation obtained from isotopic labeling was automatically generated in CPAS.

Human Subjects. For biomarker validation studies two sources of samples were relied upon. One included sera obtained at the time of diagnosis from 30 subjects with pancreatic cancer. Sera from 15 subjects with chronic pancreatitis and from 20 healthy subjects collected using the same protocol served as controls. A second source of samples included sera collected as part of the Beta-Carotene and Retinol Efficacy Trial (CARET). Thirteen subjects representing all subjects approximately a year following a blood draw and an equal number of controls were identified by CARET for the blinded pancreatic cancer marker validation study.

### Example 2

### Mouse Plasma Analysis

Plasma was sampled from mice at early and advanced stages of tumor development and from matched controls. Among the early and advanced tumor stages, 1,442 proteins were identified that were distributed across seven orders of magnitude of abundance in plasma. Comparative analysis of candidate biomarkers documented striking concordance of expression in human and mouse pancreatic tissue and in the blood from patients with pancreatic cancer relative to normal specimens. In addition to identifying markers of potential utility for pancreatic cancer diagnosis, the findings presented herein indicate that GEM models of cancer, in combination with proteomics, provide a rich source of candidate markers applicable to human cancer.

Pancreatic ductal adenocarcinoma (PDAC) is a highly lethal cancer, typically presenting at advanced stages as a disseminated and incurable disease (Hezel, et al. (2006) Genes Dev. 20:1218). PDAC is characterized by activating mutations of the Kras oncogene and inactivation of the Ink4a and ARF-p53 tumor suppressor pathways in the great majority of cases. Kras activation is thought to initiate focal lesions in the pancreatic ducts, known as pancreatic intraepithelial neoplasias (PanINs), which undergo graded histological progression to PDAC in association with subsequent Ink4a and Arf-p53 inactivation. The recent generation of mice harboring these signature genetic mutations has yielded models that closely recapitulate the histopathogenesis of the human disease with Kras^{G12D} initiating focal PanINs that rapidly undergo multistage progression in conjunction with Ink4a/Arf or p53 mutations, resulting in invasive PDAC. Importantly, these models show broadly conserved tumor biology and molecular circuitry to human PDAC. The tumors exhibit a proliferative stroma (desmoplasia) and frequent metastasis, express pancreatic ductal markers (CK-19) and apical mucins (e.g., Muc1, Muc5AC), show activation of developmental signaling pathways (Hedgehog, Notch, EGFR), and harbor syntenic genomic alterations to human PDAC (Aguirre, et al. (2003) Genes Dev. 17:3112; Bardeesy, et al. (2006) Proc. Natl. Acad. Sci. USA 103:5947; Hingorani, et al. (2005) Cancer Cell 7:469).

This faithful mouse model of human PDAC, coupled with major technological advances in proteomic science, prompted an in-depth quantitative proteomic approach to the analysis of plasma obtained from PDAC-prone mice engineered with activated Kras and Ink4a/Arf deficiency (Aguirre, et al. (2003) Genes Dev. 17:3112).

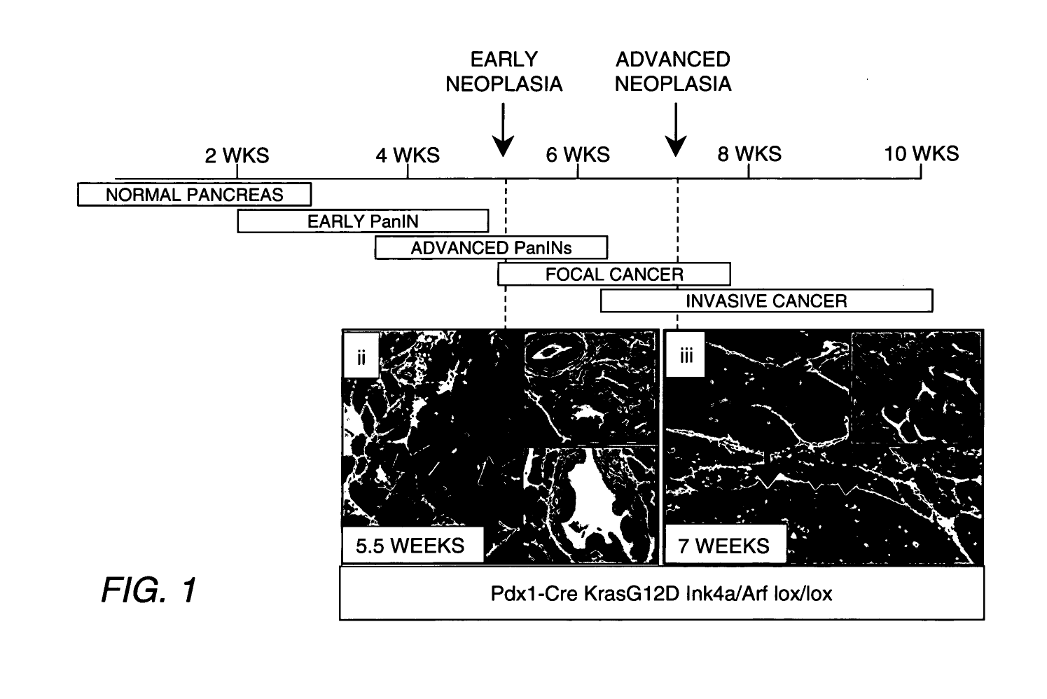

Mice harboring Pdx1-Cre Kras^{G12D} Ink4a/Arf^{lox/lox} mutations exhibit stereotypical neoplastic progression from PanIN (pancreatic cancer precursor lesions) to advanced PDAC over a narrow period from 2 to 10 weeks of age (Aguirre, et al. (2003) Genes Dev. 17:3112). A plasma pooling strategy was applied for in-depth proteomic analysis. Blood was obtained from mice at the PanIN stage and at the PDAC stage (at 5.5 and 7 weeks, respectively) and from age- and sex-matched controls, thus constituting four pools of plasma (FIG. 1). The tumor stage was confirmed for individual mice by histopathology. Differential isotopic labeling was applied to each tumor pool and its matched control (Faca (2006) J. Proteome Res. 5:2009), followed by extensive fractionation of intact proteins for in-depth quantitative analysis. Each experiment generated 156 plasma fractions based on anion exchange and reverse phase chromatography, which were analyzed separately by LC-MS/MS following tryptic digestion. Approximately 2,800,000 mass spectra were produced and analyzed. Collectively, the PanIN and PDAC experiments resulted in a primary list of 1,095 unique high confidence proteins with less than a 1% false discovery rate (FDR) based on reverse-database searches. To this primary list, 347 additional proteins were appended with less than a 5% FDR that exhibited pancreatic relevance. The criteria used for pancreatic relevance were: (i) proteins with mRNA expression in pancreas tissue greater than 2-fold compared to the mean of 61 mouse tissue expression surveys from published data (Su, et al. (2004) Proc. Natl. Acad. Sci. USA 101:6062) and/or (ii) proteins that presented high mRNA expression in pancreatic cancer compared to normal tissue, in mouse (this study) or human (Logsdon, et al. (2003) Cancer Res. 63:2649). The integration of protein identification with other biological data is intended to recognize possible biomarker candidates found in plasma at very low concentration resulting in a reduced number of peptide identifications (1 to 3 peptides). Based on UniProt keywords, 25% of identified proteins in the combined list of 1442 proteins contained a signal peptide for secretion and 20% were annotated as glycoproteins. Of note, the list included a relatively large percentage (9%) of membrane proteins based on Gene Ontology cellular component annotation. Peptides for several membrane proteins identified were derived exclusively from the extracellular domain. Epidermal growth factor receptor, for example, was detected in several fractions with peptides spanning amino acids 25 to 647 representing the extracellular N-terminal domain. These results are consistent with shedding of extracellular domains into the circulation (Hood, et al. (2005). J. Proteome Res. 4:1561).

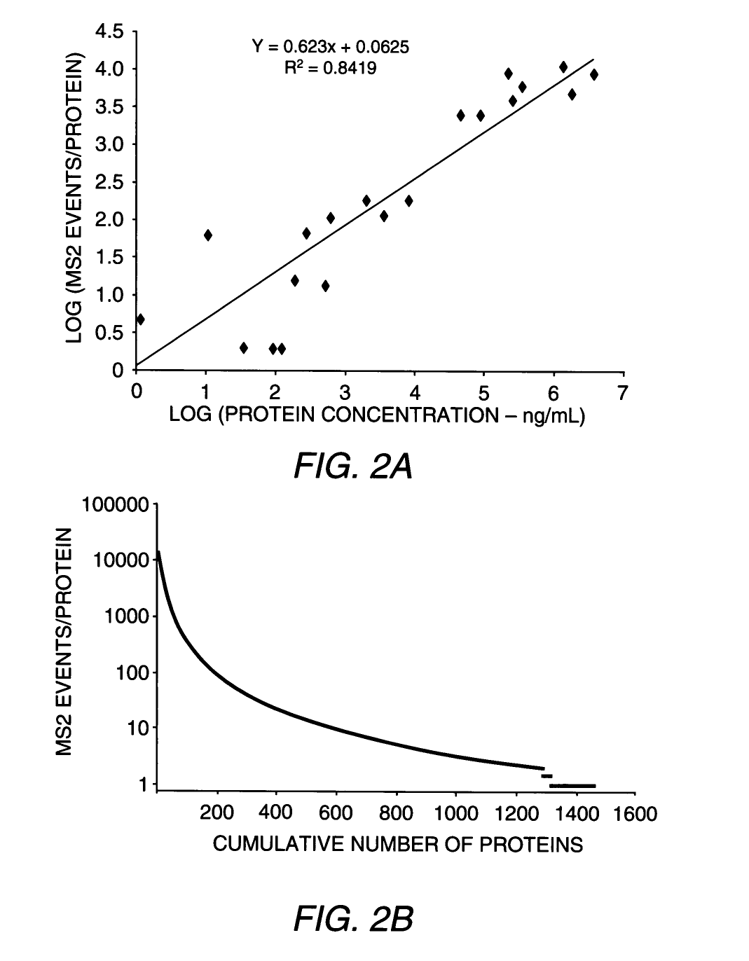

To estimate the concentration range of mouse plasma proteins identified, the method of spectral sampling was used to provide an estimation of protein abundance based on number of spectra acquired for a given protein (Liu, et al. (2004) Anal. Chem. 76:4193). The spectral sampling data (number of MS2 events/protein) was further correlated to mouse plasma proteins whose concentrations are known (Rules-Based Medicine Inc., 3300 Duval Road, Austin, Tex.). A significant correlation was observed between the number of MS2 events for a given protein and plasma protein concentration (R²=0.84) (FIG. 2A). From this correlation, it was estimated that the fractionation allowed for identification of plasma proteins across seven orders of magnitude and detection of proteins at a plasma concentration as low as 1 ng/ml. The number of proteins identified was greater at lower predicted plasma concentrations based on number of MS2 events (FIG. 2B).

The majority of medium-to-high abundance proteins were detected in both PanIN and PDAC experiments, while most differences in protein identifications between the two experiments represented lower abundance proteins. Likewise, in duplicate LC-MS/MS analysis of the same fractions, most differences in protein identifications observed represented lower abundance proteins. Experiments in which independent replicates of samples were analyzed resulted in 60% of protein sampling/identification in both experiments. Thus, differences in protein identifications between the two experiments were largely attributable to mass spectrometry sampling effects during data collection and partially to the occurrence of some proteins at a higher level of abundance in the PDAC stage compared to PanIN. Importantly, since cancer and control samples were analyzed together after isotopic labeling followed by mixing, variations related to fractionation and sample processing were minimized.

A significant proportion of plasma proteins were synthesized in the liver. While particular forms of these proteins may correlate with a specific type of cancer through cleavage or other types of modifications, unless such modifications can be singled out, overall changes in abundance levels of liver proteins are unlikely to yield cancer markers specific to the pancreas. To distinguish between such classical plasma proteins from proteins that may be derived from the pancreas in this dataset, the 1,442 proteins identified in these analyses were cross-referenced with two recent publications on proteomic profiling of mouse liver tissue (Foster, et al. (2006) Cell 125:187; Kislinger, et al. (2006) Cell 125:173). Approximately 38% of the 1,442 proteins were identified in mouse liver tissue, composed mostly of relatively abundant plasma proteins. In contrast, proteins estimated to be of low abundance in the protein list had a much greater representation of pancreatic proteins relative to liver proteins based on tissue protein and/or mRNA data.

### Example 3

### Biomarker Candidates

Acrylamide isotopic labeling of cysteine residues was used to obtain relative quantitative information between disease and control samples. This labeling approach is chemically very efficient as evidenced by lack of unlabeled cysteines in searching mass spectra (Faca (2006) J. Proteome Res. 5:2009). Additionally, this labeling chemistry is fully compatible with the intact protein approach, without significantly affecting protein physical-chemical characteristics. In duplicate experiments performed with independent replicates of samples, there were no proteins that showed quantitative inconsistencies (up-regulated in one experiment and down-regulated in the other). An important aspect of this approach is that identification is not limited to cysteine-containing peptides, thus providing a comprehensive list of peptides in the digests. Among the 626 quantified proteins, 173 were found to be up-regulated in cancer samples (PDAC or PanIN or both) compared to controls.

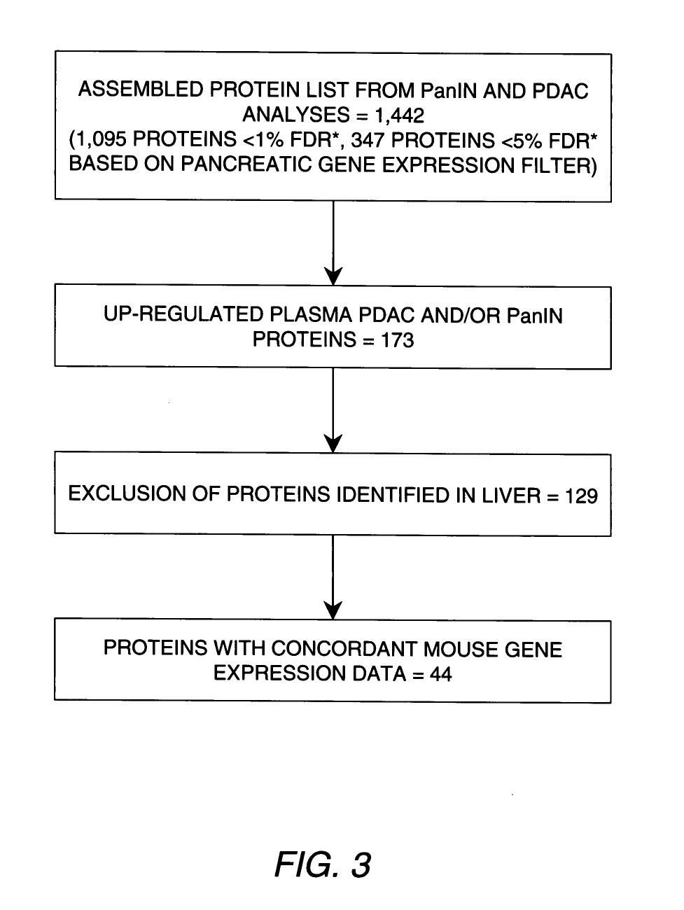

A subset of 44 (Table 1) from the 173 upregulated proteins was deemed potentially relevant to human pancreatic cancer based on the following criteria: (i) mean protein ratio in neoplasm/normal plasma >1.5 (P<0.05) in PDAC and PanIN based on isotopic labeling ratios, and/or occurrence of isotopically labeled peptides in cancer samples but not in controls; (ii) not identified in prior liver tissue proteomic studies (Foster, et al. (2006) Cell 125:187; Kislinger, et al. (2006) Cell 125:173) nor known to represent acute phase reactants; and (iii) mouse protein has a corresponding ortholog gene in human (FIG. 3). Also included in this list were proteins that were similarly elevated in either PDAC or PanIN and that had evidence of increased expression of corresponding genes in pancreatic cancer for mouse (data obtained in this study) and for human (Logsdon, et al. (2003) Cancer Res. 63:2649). Exclusion of liver proteins was intended to eliminate a major source of plasma proteins whose elevated levels was unlikely to have pancreatic cancer specificity.

Unexpectedly, of the prioritized list of pancreatic cancer candidates, some 17 proteins were previously analyzed in pancreatic cancer tissue by immunohistochemistry or for a smaller number in human blood by immunoassay (see Table 1). However, these proteins have been studied independently of each other and not identified through a systematic profiling study as presented here. Some of these proteins have been described as up-regulated in pancreatic cancer tissue by an independent proteomic analysis (Chen, et al. (2005) Gastroenterology 129:1187-97. Interestingly, wap four-disulfide core domain protein 2 (HE4 or WFDC2), a promising biomarker for ovarian cancer, was found in this study to be up-regulated in mouse PDAC plasma, with concordant mRNA expression. HE4 has also been listed as upregulated at the gene and protein levels in human PDAC tissue (Goonetilleke and Siriwardena (2007) Eur. J. Surg. Oncol. 33(3):266-70), suggesting that this protein may also have relevance to pancreatic cancer.

TABLE 1

| | Protein | | | | | |
|---|---|---|---|---|---|---|
| | Quantitation^{#} | | Gene Expression | | | |
| | PDAC | PanIN | Human | | Mouse | |
| | Cancer/ | Cancer/ | Cancer/ | Pancreatitis/ | Cancer/ | Previously |
| Gene Name | Normal | Normal | Normal | Normal | Normal | Analyzed |

Increased Levels in PDAC + PanIN

| | | | | | | |
|---|---|---|---|---|---|---|
| CDSL | 2.7 | 2.1 | 0.6 | 0.7 | 0.3 | |
| CTRB1 | 6.1 | 9.1 | 0.3 | 0.3 | 0.3 | |
| IL1RAP | Cancer^{†} | Cancer^{†} | 6.4 | 4.9 | 0.4 | |
| LCN2 | 6.9 | Cancer^{†} | 13.4 | 3.8 | 39.8 | Yes¹ |
| LRG1 | 2.9 | 2.0 | — | — | 1.3 | |
| PRG4 | 10.0 | 2.5 | 1.5 | 1.6 | 0.7 | |
| REG1A | 2.3 | 3.9 | 0.3 | 0.3 | 0.4 | Yes² |
| REG3 | 5.9 | 2.8 | — | — | 0.7 | Yes² |
| SYCN | 4.2 | 5.0 | — | — | 0.1 | |
| TIMP1 | Cancer^{†} | (1.7)* | 4.1 | 3.4 | 22.0 | Yes^{1,2} |

Increased Levels in PDAC + Increased Expression of Corresponding Genes

| | | | | | | |
|---|---|---|---|---|---|---|
| ALCAM | 2.8 | (0.9)* | 1.1 | 1.1 | 2.8 | Yes³ |
| COL18A1 | Cancer^{†} | — | 1.2 | 1.1 | 13.7 | |
| COL15A1 | 1.8 | — | 1.2 | 1.4 | 5.1 | |
| CTGF | 2.0 | 0.8 | 1.2 | 3.9 | 89.6 | |
| CTSS | 2.4 | — | 3.4 | 2.8 | 16.8 | Yes³ |
| CXCL16 | 2.8 | — | — | — | 3.2 | |
| FBLN2 | 2.6 | — | 4.1 | 8.2 | 5.7 | |
| FSTL1 | 6.5 | 1.1 | 2.3 | 3.4 | 12.7 | |
| HTRA1 | Cancer^{†} | — | — | — | 2.9 | |
| ICAM1 | 1.6 | 0.9 (1.0)* | 2.2 | 1.5 | 3.9 | Yes^{1,2} |
| LIMS1 | 2.2 | — | 0.9 | 0.8 | 16.3 | |
| LTBP4 | Cancer^{†} | — | 1.7 | 1.9 | 2.2 | Yes¹ |
| LTF | 2.3 | — | 5.7 | 0.9 | 17.0 | Yes¹ |
| LYZ | 3.2 | 1.1 | 3.6 | 2.6 | 33.3 | |
| MSH6 | 2.3 | — | 1.0 | 0.9 | 3.2 | |
| PTPRG | Cancer^{†} | — | 2.8 | 2.4 | 3.8 | |
| SOD3 | 2.9 | 0.3 | 0.5 | 0.6 | 4.7 | |
| SPARCL1 | 1.6 | 0.8 | 1.0 | 1.9 | 5.7 | |
| TNC | Cancer^{†} | — | 6.6 | 6.9 | 180.0 | Yes^{1,2} |
| TNFRSF1A | 2.5 | — | 2.4 | 2.6 | 1.5 | Yes² |
| VASP | 2.3 | — | 3.8 | 2.0 | 4.2 | |
| WFDC2 (HE4) | 2.6 | — | 2.1 | 0.9 | 25.0 | Yes¹ |
| ZDHHC20 | Cancer^{†} | — | — | — | 3.4 | |

Increased Levels in PanIN + Increased Expression of Corresponding Genes

| | | | | | | |
|---|---|---|---|---|---|---|
| CD248 | 1.3 | 1.8 | — | — | 6.47 | |
| CD97 | 1.2 | 2.6 | 4.0 | 3.7 | 9.9 | Yes^{1,2} |
| CDH1 | — | 2.0 | 1.9 | 0.8 | 2.6 | |
| EFEMP2 | 1.2 | 3.1 | 2.1 | 3.4 | 30.0 | Yes³ |
| EFNA1 | 1.0 | Cancer^{†} | 2.3 | 1.9 | 2.6 | |
| GKN1 | 1.6 | Cancer^{†} | — | — | 12.9 | |
| IGFBP4 | 1.3 | 1.5 | 2.7 | 5.2 | 37.5 | |
| SLPI | 1.9 | 2.3 | 3.9 | 1.5 | 1.9 | |
| TFF2 | — | 3.3 | 4.0 | 4.6 | 0.1 | Yes¹ |
| TGFBI | — | 2.4 | 3.2 | 4.7 | 15.9 | Yes² |
| THBS1 | 1.2 | 3.1 | 2.5 | 5.9 | 2.6 | Yes^{1,3} |

*Measurements of protein ration performed by ELISA for the same mouse plasma samples used in the proteomic analysis.
^{#}P-value for all ratios <0.05
^{†}Cancer Only.
¹Immunohistochemistry,
²Enzyme-linked immunosorbent assays,
³Proteomics.

An independent validation of the proteomic approach was conducted by measuring protein levels in mouse pancreatic tissue and in mouse plasma, based on availability of suitable antibodies. Immunohistochemical analysis (IHC) was done for CD166 antigen precursor (ALCAM), receptor-type tyrosine-protein phosphatase gamma precursor (PTPRG), tissue inhibitor of metalloproteinase 1 (TIMP1), and tenascin C (TNC). All these proteins demonstrated strong IHC staining in mice PanIN and pancreatic cancer tissue sections (FIG. 4A). Circulating protein levels of ALCAM, TIMP1 and ICAM1 (intercellular adhesion molecule 1) in the same mouse plasma used in the proteomic approach were measured by Enzyme-linked immunosorbent assays (ELISA). ALCAM, TIMP1 and ICAM1 had significantly higher levels in PDAC mice plasmas. TIMP1 was significantly elevated in PanIN plasma samples as well.

### Example 4

### Biomarker Validation in Human Samples

To assess the relevance of these candidate markers identified in the mouse model of pancreatic cancer to human pancreatic cancer, validation studies were undertaken in human tissue and/or blood samples based on availability of suitable antibodies. Immunohistochemistry was performed for PTPRG, TNFRSF1a (tumor necrosis factor receptor superfamily member 1a precursor) and TNC, all of which showed positive IHC staining in human pancreatic cancer (FIG. 4B).

Enzyme-linked immunosorbent assays (ELISA) applicable to human samples were available for ALCAM, ICAM1, LCN2 (neutrophil gelatinase-associated lipocalin), sTNFRSF1, TIMP1, REG1A (lithostathine 1), REG3 (regenerating islet-derived protein 3), HE4 and IGFBP4 (insulin-like growth factor binding protein 4). These candidates were assayed in human sera from 30 patients with PDAC to assess their significance individually and as a panel, together with CA19.9, a marker which is currently in clinical use as a pancreatic cancer marker (Goonetilleke and Siriwardena (2007) Eur. J. Surg. Oncol. 33(3):266-70) (Table 2). As a control group, sera from 20 matched healthy subjects and 10-15 subjects with chronic pancreatitis were analyzed. Statistical analysis was performed for individual markers and for the entire panel as a group. All but one of the candidate markers were significantly elevated in cancer compared to one or both control groups (P-value<0.03). Seven candidates were compared between cancer and both control groups and five of seven candidates were significant in both comparisons (P-value<0.03) (Table 2). Only one candidate (LCN2) did not achieve statistical significance. For candidate markers that yielded statistically significant differences between cancer and healthy subjects the areas under the curve (AUCs) ranged between 0.75 and 0.89, and between cancer and pancreatitis the AUCs ranged between 0.74 and 0.92 (Table 3). Of note, a panel of all the candidates inclusive of the candidate that did not achieve statistical significance individually to avoid any overfitting, yielded an AUC of 0.96 in contrast to CA 19.9 which yielded an AUC of 0.79 (FIG. 5).

TABLE 2

| | | | Healthy* | | | |
|---|---|---|---|---|---|---|
| | Cancer* | | | Std | Pancreatitis* | |
| Biomarker | Mean | Std Dev. | Mean | Dev. | Mean | Std Dev. |
| CA19.9 (U/ml) | 147.7 | 75.6 | 14.7 | 17.0 | 65.0 | 82.0 |

Concentration in Human Serum (ng/ml)

| | | | | | | |
|---|---|---|---|---|---|---|
| ALCAM | 139.9 | 65.9 | 84.2 | 26.0 | 96.4 | 30.6 |
| TIMP1 | 322.7 | 196.0 | 182.3 | 33.7 | 178.3 | 39.3 |
| ICAM1 | 457.4 | 384.8 | 270.7 | 67.8 | 121.4 | 64.9 |
| LCN2 | 162.6 | 83.8 | 138.6 | 49.2 | 120.3 | 26.8 |
| REG1A | 1108.4 | 624.9 | 691.3 | 342.2 | 1277.6 | 1219.5 |
| REG3 | 22.6 | 15.10.07 | 8.6 | 5.1 | 11.0 | 5.9 |
| IGFBP4 | 153.5 | 125.6 | 116.1 | 74.5 | 89.6 | 29.6 |
| TNFRSF1A | 2.8 | 1.5 | 1.8 | 0.9 | — | — |
| WFDC2 (HE4) | 10.9 | 18.4 | 2.5 | 8.2 | — | — |

TABLE 3

| | Area Under the | | Wilcoxon Ranksum | |
|---|---|---|---|---|
| | Curve (AUC) | | Test (P-Value) | |
| | Cancer vs. | Cancer vs. | Cancer vs. | Cancer vs. |
| Biomarker | Healthy | Pancreatitis | Healthy | Pancreatitis |
| CA19.9 | 0.98 | 0.79 | <0.001 | 0.007 |
| ALCAM | 0.85 | 0.74 | <0.001 | 0.008 |
| TIMP1 | 0.89 | 0.88 | <0.001 | <0.001 |
| ICAM1 | 0.75 | 0.92 | 0.029 | <0.001 |
| LCN2 | 0.65 | 0.61 | 0.452 | 0.318 |
| REG1A | 0.79 | 0.57 | 0.002 | 0.528 |
| REG3 | 0.88 | 0.77 | <0.001 | 0.011 |
| IGFBP4 | 0.69 | 0.74 | 0.106 | 0.022 |
| TNFRSF1A | 0.82 | — | 0.008 | — |
| WFDC2 (HE4) | 0.89 | — | 0.001 | — |

Advantageously, a panel test of the invention can be carried out with some or all (e.g., 1, 2, 3, 4, 5, 6, 7, 8, 9, or 10) of the markers set forth in Table 2 herein.

To determine the potential of candidate markers identified at the PanIN stage for early diagnosis of pancreatic cancer, a blinded analysis was conducted using sera collected as part of the Beta-Carotene and Retinol Efficacy Trial (CARET), which included 18,314 participants (Goodman, et al. (2004) J. Natl. Cancer Inst. 96:1743-50). The CARET study was intended to test the effect of daily beta-carotene and retinyl palmitate on cancer incidence and death in subjects with a history of smoking or asbestos exposure. All subjects (13) diagnosed with pancreatic cancer between 7-13 months following a blood draw (mean=10 months) and an equal number of controls that were matched for age, sex, year of CARET enrollment and time of blood draw in relation to enrollment who were not diagnosed with pancreatic cancer based on information in the CARET database were identified by CARET for the blinded pancreatic cancer marker validation study. The pancreatic cancer and control groups were also matched for CARET intervention. Five candidate markers, for which ELISA assays were available (LCN2, REG1A, REG3, TIMP1 and IGFBP4), were assayed together with CA19.9 without knowledge of which subjects developed pancreatic cancer subsequent to the blood draw and which subjects were matched controls (Table 4). Two of the five candidate markers (IGFBP4 and TIMP1) showed significance at 0.05 and 0.04, respectively. CA19.9 was significant at 0.04. As a panel, the candidate markers tested achieved an AUC of 0.817 (p=0.005), inclusive of candidates that did not achieve statistical significance individually to avoid any overfitting. When the panel of markers was combined with CA19.9 an AUC of 0.911 was achieved (FIG. 6).

TABLE 4

| | | | | | | Wilcoxon |
|---|---|---|---|---|---|---|
| | Cancer | | Healthy | | | Ranksum |
| Bio- | | Std | | Std | Area Under | Test |
| marker | Mean | Dev. | Mean | Dev. | the Curve | (P-value) |
| CA19.9 | 56.1 | 74.2 | 10.6 | 6.7 | 0.74 | 0.040 |
| (U/ml) | | | | | | |

Concentration in Human Serum (ng/ml)

| | | | | | | |
|---|---|---|---|---|---|---|
| TIMP1 | 212.7 | 62.4 | 162.1 | 36 | 0.74 | 0.040 |
| IGFBP4 | 61 | 33.1 | 45.6 | 50.5 | 0.72 | 0.050 |
| LCN2 | 123.5 | 42.6 | 104.4 | 66.1 | 0.68 | 0.110 |
| REG3 | 25.2 | 21.7 | 17.2 | 14.3 | 0.65 | 0.220 |
| REG1 | 2111.4 | 1151.9 | 2059.1 | 1325.1 | 0.52 | 0.980 |

The depth of proteomic analysis achieved in this study allowed the identification and quantitative analysis of low abundance proteins and uncovered a large number of protein changes in mouse plasma with pancreatic tumor development, some corresponding to previously observed changes in human pancreatic tumor tissue at the gene expression or protein level and others representing novel findings.

The foregoing is illustrative of the present invention, and is not to be construed as limiting thereof. The invention is defined by the following claims, with equivalents of the claims to be included therein.

1. A method of diagnosing, prognosing or screening for pancreatic cancer in a subject, comprising:
(a) detecting one or more markers in a biological sample of said subject, said markers selected from the group consisting of ALCAM, ICAM1, LCN2, MMP2, sTNFRSF1, TIMP-1, IGFBP4, Reg3, Reg3g, Reg1, Reg1a, Sycn, Itsn2, Rnase 1, CD248, Loxl1, Avp, Efemp2, Clec4f, and cleavage products thereof; and
(b) determining altered levels of said marker(s), said altered levels indicating said subject may be afflicted with or at risk of developing pancreatic cancer.

2. The method of claim 1, wherein said one or more markers includes ALCAM.

3-18. (canceled)

19. The method of claim 1, wherein said one or more markers are included in a diagnostic panel comprising at least two of ALCAM, ICAM1, LCN2, MMP2, and TIMP-1.

20. The method of claim 1, wherein said one or more markers are included in a diagnostic panel consisting of ALCAM, ICAM1, LCN2, MMP2, and TIMP-1.

21-22. (canceled)

23. The method of claim 1, wherein said sample is a tumor sample.

24. The method of claim 1, wherein said sample is a blood sample.

25-27. (canceled)

28. The method of claim 1, wherein said altered levels are compared to a control sample, with said control sample taken from the same or different subject.

29. The method of claim 1, wherein said subject is afflicted with pancreatic cancer.

30. The method of claim 1, wherein said detecting step is carried out by immunoassay, chromatography, spectrometry, electrophoresis, sedimentation, isoelectric focusing, or any combination thereof.

31-34. (canceled)

35. A kit comprising one or more means of detecting one or more markers of pancreatic cancer in a subject in determining if said subject may be afflicted with or at risk of developing pancreatic cancer, said markers selected from the group consisting of ALCAM, ICAM1, LCN2, MMP2, sTNFRSF1 and TIMP-1, Reg3g, Reg1, Sycn, Itsn2, Rnase 1, CD248, Loxl1, Avp, Efemp2, Clec4f, and cleavage products thereof.

36. The kit of claim 35, wherein said one or more markers includes ALCAM.

37-52. (canceled)

53. The kit of claim 35, wherein said one or more markers are included in a diagnostic panel comprising at least two of ALCAM, ICAM1, LCN2, MMP2, and TIMP-1.

54. The kit of claim 35, wherein said one or more markers are included in a diagnostic panel consisting of ALCAM, ICAM1, LCN2, MMP2, and TIMP-1.

55-57. (canceled)

58. The kit of claim 35, wherein said one or more means of detecting is carried out by immunoassay, chromatography, spectrometry, electrophoresis, sedimentation, isoelectric focusing, or any combination thereof.

59. (canceled)

60. The method of claim 1, wherein said subject has previously been diagnosed as afflicted with pancreatic cancer.

61. The method of claims claim 1, wherein said subject has previously been diagnosed as afflicted with pancreatic cancer and has previously been treated for said pancreatic cancer.

62. A method of treating pancreatic cancer in a subject in need thereof, comprising:
administering said subject a therapeutic antibody in an amount effective to treat said cancer, wherein said therapeutic antibody specifically binds to a marker selected from the group consisting of ALCAM, ICAM1, LCN2, MMP2, sTNFRSF1, TIMP-1, Reg3g, Reg1, Sycn, Itsn2, Rnase 1, CD248, Loxl1, Avp, Efemp2, and Clec4f.

63. The method of claim 62, wherein said antibody is a monoclonal antibody.

64. (canceled)

65. The method of claim 1, further comprising detecting an altered level of an additional marker in said biological sample, wherein said additional marker is CA19.9; wherein altered levels of both (i) said one or more markers and (ii) said additional marker, indicate said subject may be afflicted with or at risk of developing pancreatic cancer.

66. The method of claim 65, wherein said one or more marker is selected from the group consisting of LCN2, REG1A, REG3, TIMP1, and IGFBP4.
